(19) United States
(12) Patent Application Publication
(10) Pub. No.: US 2013/0158353 A1
(43) Pub. Date: Jun. 20, 2013

(54) PROPULSION ASSEMBLY FOR ENDOSCOPE

(71) Applicant: FUJIFILM Corporation, Tokyo (JP)

(72) Inventors: Takayuki NAKAMURA, Ashigarakami-gun (JP); Tsuyoshi ASHIDA, Ashigarakami-gun (JP); Nobuyuki TORISAWA, Ashigarakami-gun (JP); Takumi DEJIMA, Ashigarakami-gun (JP); Naoyuki MORITA, Ashigarakami-gun (JP)

(73) Assignee: FUJIFILM Corporation, Tokyo (JP)

(21) Appl. No.: 13/714,069

(22) Filed: Dec. 13, 2012

(30) Foreign Application Priority Data
Dec. 16, 2011 (JP) 2011-275311

(51) Int. Cl. A61B 1/00; A61B 1/06; A61B 1/005; A61B 1/31; A61B 1/018; A61B 1/07; A61B 1/05; A61B 1/273

(52) U.S. Cl. CPC A61B 1/00156; A61B 1/05; A61B 1/0676; A61B 1/00066; A61B 1/2736; A61B 1/31; A61B 1/051; A61B 1/018; A61B 1/00117; A61B 1/07; A61B 1/00009; A61B 1/0057
USPC 600/114

(57) ABSTRACT

A propulsion assembly for an endoscope includes a support sleeve and a barrel unit. An endless track device is disposed to extend along inner and outer surfaces of the barrel unit, for endlessly moving in an axial direction of an elongated tube of the endoscope, and contacting a wall of a body cavity, for propulsion of the elongated tube. Worm wheels are disposed on the support sleeve, for driving the endless track device by engagement therewith. Plural idler rollers are disposed on the inner surface of the barrel unit in a rotatable manner, for keeping the endless track device movable in driving of the worm wheels. An guide projection is formed on the endless track device. A guide groove is formed in the barrel unit, for receiving the guide projection, to guide the endless track device on the barrel unit in the axial direction.

### BACKGROUND OF THE INVENTION

1. Field of the Invention

The present invention relates to a propulsion assembly for an endoscope. More particularly, the present invention relates to a propulsion assembly for an endoscope, in which friction of an endless track device with a wall of a body cavity can be reduced and damage of the endless track device can be prevented.

2. Description Related to the Prior Art

An endoscope is widely used for medical diagnosis of a body of a patient. The endoscope has an elongated tube for entry in a body cavity of the body. The elongated tube includes a tip device, a steering device and a flexible tube portion. The steering device operates for orienting the tip device in a suitable direction. An imaging window is disposed at an end face of the tip device for receiving light from an object for imaging.

In the diagnosis, entry of the endoscope into a large intestine is technically difficult due to a tortuous form of the large intestine with portions highly movable in the body. A doctor or operator of the diagnosis must have high skill in manipulating the endoscope. Recently, a propulsion assembly for the endoscope has been proposed. The propulsion assembly propels the endoscope in the body cavity in an axial direction and facilitates the entry of the endoscope even for a doctor or operator before having suitable skill of the manipulation.

U.S. Pat. Nos. 6,971,990 and 7,736,300 (corresponding to JP-A U. S 2009-513250) discloses the propulsion assembly, which includes a support sleeve, an endless track device, a barrel sleeve and a drive unit. The support sleeve is mounted on the elongated tube of the endoscope. The endless track device is a toroidal device constituted by a flexible membrane, and extends around the support sleeve according to the axial direction of the elongated tube. The barrel sleeve is disposed inside the endless track device and supports the endless track device. The drive unit endlessly moves the endless track device in contact with an inner wall of the body cavity, to move the elongated tube back or forth in the axial direction.

According to U.S. Pat. Nos. 6,971,990 and 7,736,300 (corresponding to JP-A 2009-513250), the endless track device of the propulsion assembly endlessly moves in tight contact with a peripheral surface of the barrel sleeve. High friction is likely to occur to create considerable stress to the endless track device. Durability of the endless track device will be low according to its damage due to torsion. Also, load to the drive unit for moving the endless track device may be increased extremely.

### SUMMARY OF THE INVENTION

In view of the foregoing problems, an object of the present invention is to provide a propulsion assembly for an endoscope, in which friction of an endless track device with a wall of a body cavity can be reduced and damage of the endless track device can be prevented.

In order to achieve the above and other objects and advantages of this invention, a propulsion assembly for an endoscope includes a support sleeve for mounting on a tip portion of an elongated tube of the endoscope. A barrel unit is disposed around the support sleeve. An endless track device is disposed to extend along inner and outer surfaces of the barrel unit, for endlessly moving in an axial direction of the elongated tube in contact with a wall of a body cavity, for propulsion of the tip portion. A plurality of roller wheels are disposed on the support sleeve, for driving the endless track device by engagement therewith. A plurality of idler rollers are disposed on the inner surface of the barrel unit in a rotatable manner, for applying tension to the endless track device in cooperation with the roller wheels. At least one guide projection is formed on a first one of the endless track device and the barrel unit. At least one guide groove is formed in a second one of the endless track device and the barrel unit, for receiving the guide projection, to guide the endless track device on the barrel unit in the axial direction.

Furthermore, a drive sleeve is supported around the support sleeve, for rotating upon application of torque. Worm gear teeth are formed around the drive sleeve, for rotating the roller wheels.

The roller wheels have helical gear teeth.

The guide projection is formed on the endless track device, and the guide groove is formed in the barrel unit.

The guide projection contacts the inner and outer surfaces of the barrel unit with a small contact width to prevent a stiction phenomenon of the endless track device to the inner and outer surfaces.

The barrel unit includes proximal and distal end surfaces, positioned opposite to one another with reference to the axial direction, for inverting the endless track device. The guide groove is formed in at least one of the proximal and distal end surfaces.

The barrel unit includes a barrel sleeve, and a support ring, attached to one end of the barrel sleeve in the axial direction, having the proximal or distal end surface, the guide groove being formed therein.

A depth of the guide groove is smaller than a height of the guide projection.

Furthermore, a roller groove is formed in each of the idler rollers, for receiving the guide projection.

In another preferred embodiment, the at least one guide groove has plural guide groove openings arranged in series.

In still another preferred embodiment, the guide projection is formed on the barrel unit, and the guide groove is formed in the endless track device.

The wheel includes plural gear teeth. Furthermore, plural engaging teeth are formed to project from an outer surface of the endless track device, arranged in the axial direction, for mesh with the gear teeth.

The endless track device is toroidal.

Consequently, friction of an endless track device with a wall of a body cavity can be reduced and damage of the endless track device can be prevented, because the groove and the guide projection can cooperate for aligning the endless track device reliably.

### BRIEF DESCRIPTION OF THE DRAWINGS

The above objects and advantages of the present invention will become more apparent from the following detailed description when read in connection with the accompanying drawings, in which:

FIG. 1 is an explanatory view illustrating an endoscope and a propulsion assembly in combination;

FIG. 2 is a perspective view illustrating the propulsion assembly of which an endless track device is developed;

FIG. 3 is an exploded perspective view illustrating the propulsion assembly;

FIG. 4 is an exploded perspective view illustrating a drive sleeve, torque wire devices and motors;

FIG. 5 is a vertical section illustrating the propulsion assembly;

FIG. 6 is an explanatory view in a cross section illustrating the endless track device;

FIG. 7 is a cross section taken on line VII-VII in FIG. 5;

FIG. 8 is a cross section taken on line VIII-VIII in FIG. 5;

FIG. 9 is a cross section, partially broken, illustrating another preferred set of a guide projection and a groove having a depth smaller than a height of the projection;

FIG. 10 is a cross section, partially broken, illustrating one preferred set of a guide projection formed on a support ring and a groove formed in the endless track device;

FIG. 11 is a perspective view illustrating still another preferred embodiment with plural grooves formed in the endless track device.

### DETAILED DESCRIPTION OF THE PREFERRED EMBODIMENT(S) OF THE PRESENT INVENTION

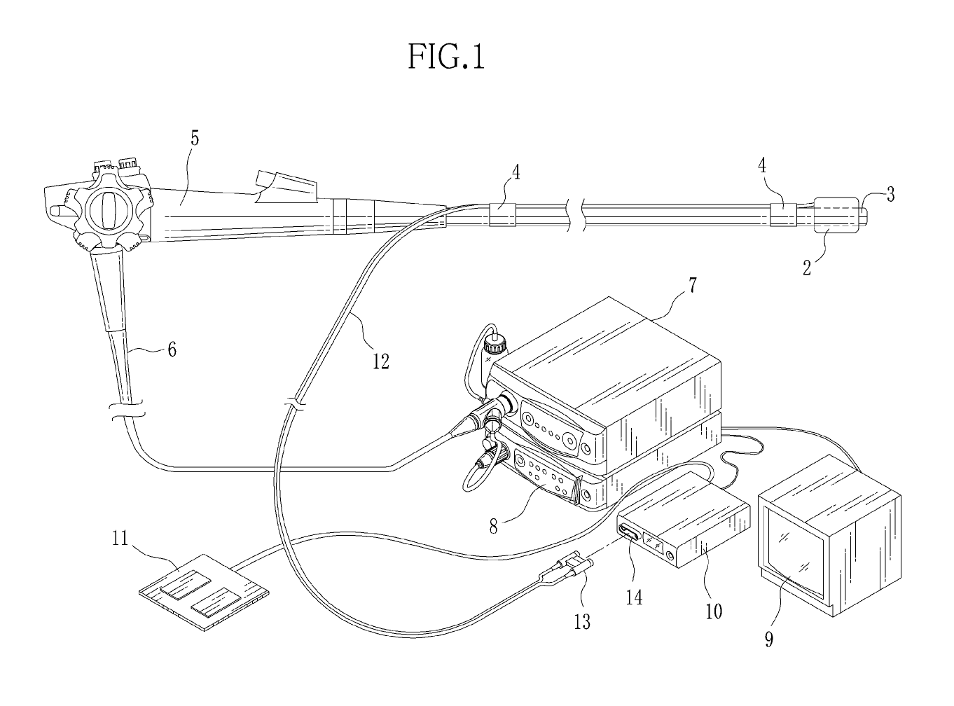

In FIG. 1, a propulsion assembly 2 is for use with an endoscope. The propulsion assembly 2 is fitted around a tip device 3 of the endoscope. The endoscope includes an image sensor, lighting windows, a steering device, an elongated tube, a handle 5, steering wheels and the like. The image sensor is incorporated in the tip device 3, and is a CCD or CMOS image sensor. The lighting windows are formed in the tip device 3 and emit light. The image sensor images an object in a body cavity illuminated with the light from the lighting windows, such an object as a wall of a stomach or intestine of a gastrointestinal tract of a patient. The steering device is disposed at a proximal end of the tip device 3 for steering to enter the tip device 3 in the body cavity to reach the object. The propulsion assembly 2 operates to facilitate the entry of the tip device 3. The steering wheels are disposed on the handle 5, and manually rotated to operate the steering device for bending up and down and to the right and left.

The handle 5 includes a button and an end sleeve. The button is operable to change over the supply and suction of air or water. The end sleeve has an instrument opening where a biopsy forceps or other medical device is advanced. A universal cable 6 extends from the handle 5, and connected to a light source apparatus 7 and a processing apparatus 8. Light from a lamp in the light source apparatus 7 is guided by a light guide fiber extending through the universal cable 6 and the endoscope to the lighting windows. The processing apparatus 8 processes an image signal from the universal cable 6 in the signal processing suitably. A display panel 9 is driven to display the image of the image signal.

The processing apparatus 8 discerns the type information of the endoscope for use according to the input information from the endoscope through the universal cable 6. The processing apparatus 8 automatically changes over the control and/or display suitably according to the type information, typically if the control with differences for the types is required in the course of the manipulation, or if the display with differences for the types is required on the display panel 9.

An actuating apparatus 10 or controller is connected with the processing apparatus 8 electrically. The actuating apparatus 10 actuates and controls the propulsion assembly 2. A wire sheath 12 of a dual lumen form extends from a proximal end of the propulsion assembly 2. An adhesive tape 4 or surgical tape positions the wire sheath 12 on the elongated tube of the endoscope at suitable points. The wire sheath 12 extends properly into the body cavity even upon moving the endoscope into the body cavity or during the manipulation.

First and second torque wire devices are disposed to extend discretely through the wire sheath 12. Distal end portions of the wire devices are coupled to a driving mechanism (sleeve) of the propulsion assembly 2. The wire devices are flexible but have high torsional rigidity so that torque applied to their proximal end are transmitted by those to their distal end substantially without attenuation. A key coupling device 13 for plug-in is disposed at the proximal end of the wire devices. A rotating coupling 14 for plug-in is disposed in the actuating apparatus 10, and connected mechanically with the key coupling device 13. First and second motors are incorporated in the actuating apparatus 10. When the key coupling device 13 is plugged to the rotating coupling 14, each of the wire devices is ready to rotate with one of the first and second motors.

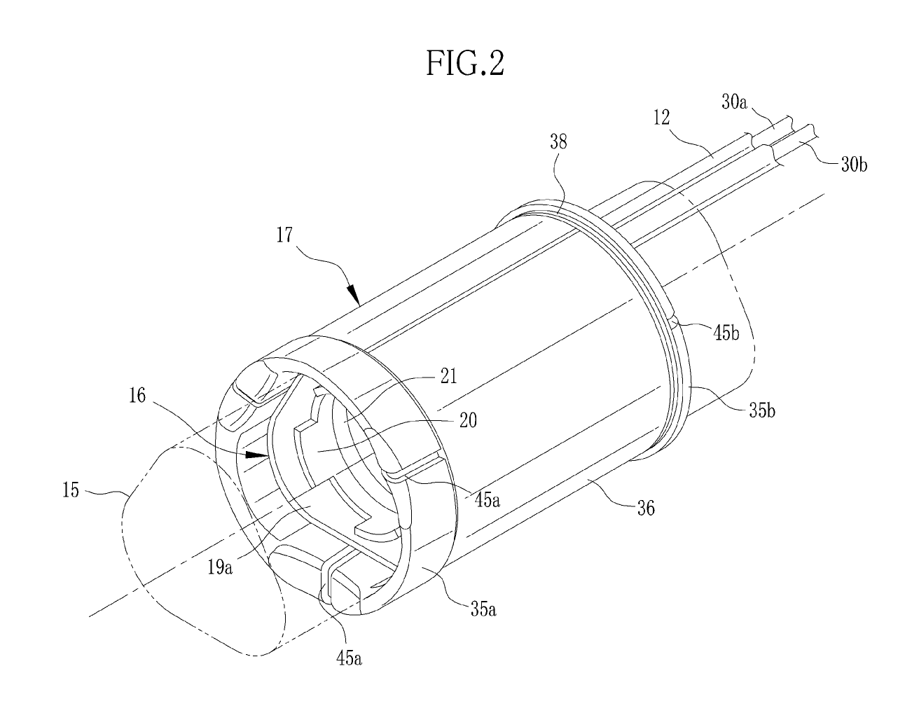

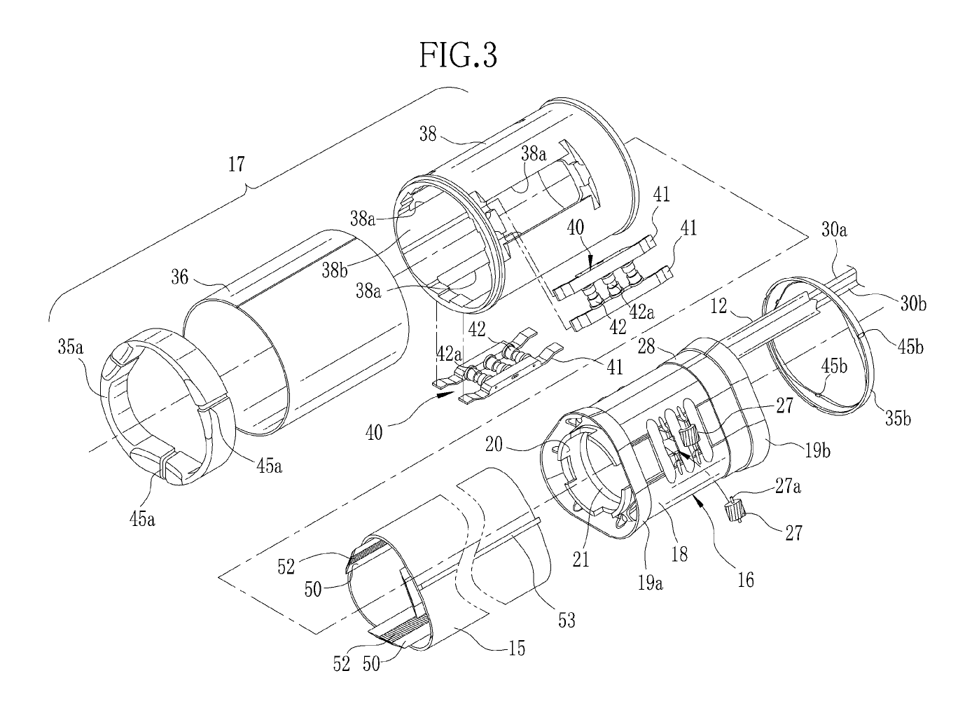

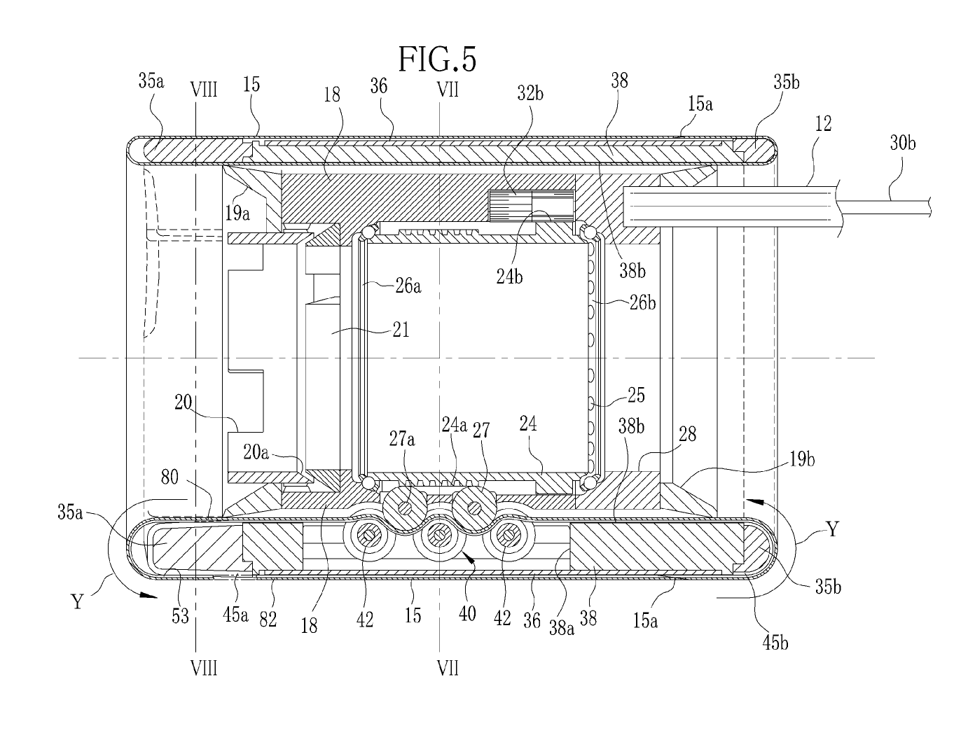

The propulsion assembly 2 is used effectively specially for colonoscopy, because of manipulation for advance and pull in the sigmoid colon or transverse colon. The propulsion assembly 2 is substantially cylindrical. An endless track device 15 or membrane or toroidal device is disposed on the outside of the propulsion assembly 2, is constituted by a flexible sheet of synthetic resin with sufficient rigidity. In FIGS. 2 and 3, the endless track device 15 is depicted in a developed form of a sleeve for understanding. A final form of the endless track device 15 is in a ring shape or toroidal shape after connecting front and rear ends of the sleeve. The endless track device 15 has an annular surface. See FIG. 5. In FIGS. 2-5, a distal side for protruding the tip device 3 is depicted on the left side. A proximal side near to the handle 5 of the endoscope is depicted on the right side.

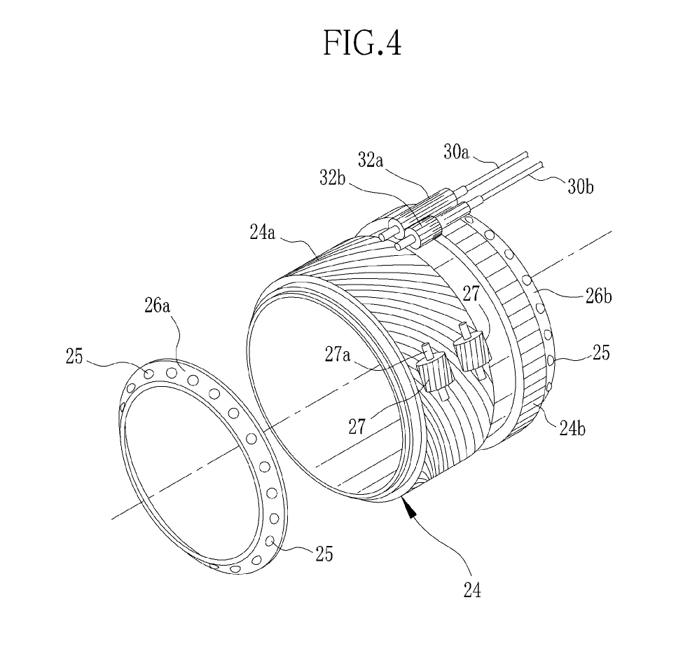

In FIGS. 2 and 3, the propulsion assembly 2 includes a drive unit 16 or inner unit, and a barrel unit 17 or outer unit. The drive unit 16 is disposed inside the endless track device 15. The barrel unit 17 is disposed around the drive unit 16. The drive unit 16 includes a support sleeve 18, a cap ring 28, a distal cover flange 19a for wiping, a proximal cover flange 19b for wiping, a clamping sleeve 20 or collet sleeve, a sealing device 21 (in a C-shape) or C-ring or collet head, and a drive sleeve 24 of FIG. 4. The support sleeve 18 has a cylindrical inner surface and an outer surface in a shape of a triangular prism. The cap ring 28 is in a triangular shape, and retained to a proximal end of the support sleeve 18 by a screw, press-fit or caulking. The cover flanges 19a and 19b are attached to respectively the distal end of the support sleeve 18 and the proximal end of the cap ring 28. The clamping sleeve 20 is helically engaged with a thread formed inside the support sleeve 18, and rotates to move in the axial direction. The sealing device 21 is formed from synthetic resin with resiliency, and has a diameter changeable by movement of the clamping sleeve 20 in the axial direction. The drive sleeve 24 is a driving mechanism supported inside the support sleeve 18 in a rotatable manner.

In FIG. 4, the propulsion assembly 2 includes bearing rings 26a and 26b, each of which is constituted by plural bearing balls 25 arranged annularly. The bearing rings 26a and 26b support ends of the drive sleeve 24 on an inner surface of the support sleeve 18 in a rotatable manner. The cap ring 28 is secured to a proximal end of the support sleeve 18, and prevents the drive sleeve 24 from dropping out. Worm gear teeth 24a or thread, and spur gear teeth 24b are arranged on an outer surface of the drive sleeve 24. Two rotatable roller wheels 27, or worm wheels with helical gear teeth for driving, are supported on the support sleeve 18, and meshed with the worm gear teeth 24a through openings in the support sleeve 18. Three pairs of the roller wheels 27 are arranged equiangularly from one another around the drive sleeve 24. When the drive sleeve 24 rotates, the roller wheels 27 rotate around a gear shaft 27a in the same direction simultaneously.

A distal end of the wire sheath 12 is attached to the inside of the proximal end of the cap ring 28 by use of adhesion or thermal welding. Distal ends of first and second torque wire devices 30a and 30b protruding from the wire sheath 12 extend to pass through holes in the cap ring 28. First and second coupling gears 32a and 32b or pinions are firmly connected with distal ends of the wire devices 30a and 30b. As illustrated in the drawing, rotational shafts protrude from respectively the coupling gears 32a and 32b as rotational centers. The shafts are received in holes formed in the support sleeve 18, to keep the coupling gears 32a and 32b rotatable. Only the first coupling gear 32a of the first wire device 30a is meshed with the spur gear teeth 24b of the drive sleeve 24.

The second coupling gear 32b of the second wire device 30b is meshed with the first coupling gear 32a but not with the spur gear teeth 24b. Thus, the drive sleeve 24 is driven by rotation of the first coupling gear 32a in connection with the first wire device 30a. However, the wire devices 30a and 30b are driven by torques generated by respectively the motors. The second coupling gear 32b is rotated in a direction opposite to that of the first coupling gear 32a. The torque from the second wire device 30b is added to the torque of the first coupling gear 32a, so that the drive sleeve 24 can be rotated with a high torque.

Each of the cover flanges 19a and 19b includes a flange edge shaped to increase a width in the axial direction. The flange edge receives an inner surface of the endless track device 15 with closeness while the endless track device 15 turns around. The flange edge prevents various materials from pull into the propulsion assembly 2 together with the moving outer surface of the endless track device 15, the materials including foreign material and tissue of a body part.

A distal end of the clamping sleeve 20 has a pattern of projections and recesses arranged in the circumferential direction. A special screw driving device for the clamping sleeve 20 is entered for engagement with the clamping sleeve 20 in the proximal direction. The clamping sleeve 20 is rotated in a predetermined direction by the screw driving device, and thus shifts in the proximal direction. A tapered end surface 20a of the clamping sleeve 20 in FIG. 5 presses the sealing device 21, which deforms to decrease the diameter. Accordingly, an inner surface of the sealing device 21 is strongly pressed on a peripheral surface of the tip device 3 for firmly fitting the support sleeve 18 thereon.

The barrel unit 17 includes a distal support ring 35a or bumper ring, a cover sheet 36 for shielding, a barrel sleeve 38 for supporting rollers, and a proximal support ring 35b or bumper ring, in a sequence in the proximal direction. The barrel unit 17 is combined with the drive unit 16 and the endless track device 15 according to the steps as follows.

In FIGS. 2 and 3, a sheet material for the endless track device 15 in a developed form is formed in a cylindrical shape. The drive unit 16 is positioned so that its outer surface is covered inside the cylindrical shape of the sheet material. The drive unit 16 with the endless track device 15 is entered in the barrel sleeve 38. Three holder openings 38a are formed in the barrel sleeve 38 to extend in the axial direction, and arranged equiangularly from one another with 120 degrees. Roller mechanisms 40 are mounted in respectively the holder openings 38a.

The roller mechanisms 40 include three idler rollers 42, and a pair of roller supports 41 or frames for supporting the idler rollers 42 in alignment. The roller supports 41 are resilient thin plates of metal, and are fixed to the barrel sleeve 38 by fitting their ends in end portions of the holder openings 38a. A center of the roller supports 41 in the longitudinal direction becomes curved to enter an inner space in the barrel sleeve 38 through the holder openings 38a. The idler rollers 42 supported by the roller supports 41 press the endless track device 15 toward the roller wheels 27 owing to the curved form of the roller supports 41.

As a result, the endless track device 15 is tensioned tightly between the roller wheels 27 and the idler rollers 42. See FIG. 5. Two of the roller wheels 27 and three of the idler rollers 42 are alternate with one another to constitute one array for running the endless track device 15 with tension. Three of such arrays are arranged equiangularly around the axis of the drive and barrel units 16 and 17. There is degree of freedom in one of the idler rollers 42 disposed at the center in relation to the longitudinal direction of the roller supports 41, because the center roller is supported by the opening extending longitudinally. A relative position of the endless track device 15 to two lateral rollers included in the idler rollers 42 is automatically adjusted for supporting the endless track device 15 with the roller wheels 27 in an optimally balanced manner.

The roller mechanisms 40 are fitted in the holder openings 38a fixedly on the barrel sleeve 38. The idler rollers 42 project to the inside of the barrel sleeve 38 and keep the barrel sleeve 38 immovable in the axial direction relative to the drive unit 16. The endless track device 15 is tensioned while the roller mechanisms 40 are combined with the barrel sleeve 38. The support rings 35a and 35b are fixed to respectively the distal and proximal ends of the barrel sleeve 38. Three guide grooves 45a are formed in the distal support ring 35a. Three guide grooves 45b are formed in the proximal support ring 35b. The guide grooves 45a and 45b are aligned with the roller mechanisms 40 in the axial direction.

The cover sheet 36 tightly covers the outer surface of the barrel sleeve 38 together with the roller mechanisms 40. The sleeve of the endless track device 15 in a developed form is positioned between the drive and barrel units 16 and 17. Those units are combined with one another, before ends of the sleeve of the endless track device 15 are turned over and connected with one another. A joint portion 15a of the endless track device 15 is formed. Note that inclinations can be preferably formed with ends of the sleeve of the endless track device 15, so that the joint portion 15a can have a small thickness without an excessive unevenness of the thickness. In FIG. 5, an assembled structure of the propulsion assembly 2 is schematically illustrated. The endless track device 15 can have an inner space to wrap the barrel unit 17 entirely in the toroidal shape. It is possible to fill the inner space with suitable fluid, such as air, physiological saline water, colloid of synthetic resin, oil, grease, lubricant fluid of various types, and the like.

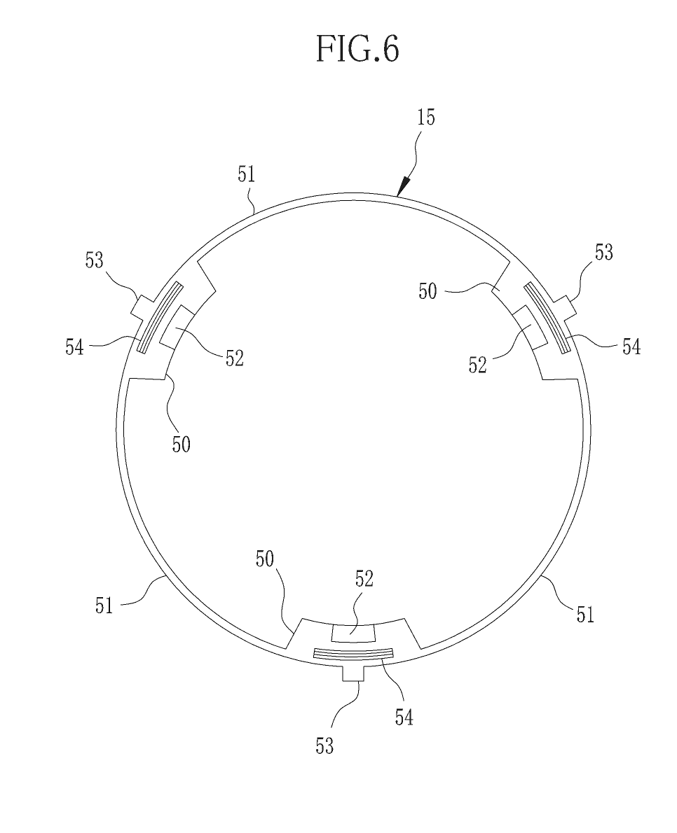

In FIG. 6, the sleeve for forming the endless track device 15 is viewed in a cross section. The endless track device 15 is constituted by a multi-layer sheet of polyurethane resin or the like with plural film layers. Three reinforcing ridges 50 are formed on an inner sleeve surface of the endless track device 15, arranged equiangularly from one another, and formed in a trapezoidal shape as viewed in section. The reinforcing ridges 50 have a larger thickness than a membrane wall 51, and are constituted by a sheet of plural film layers of a higher number than those in the membrane wall 51. The reinforcing ridges 50 extend longitudinally in the axial direction. Engaging teeth 52 or rack gear teeth are disposed on the surface of the reinforcing ridges 50, and arranged with an inclination for mesh with the roller wheels 27. Guide projections 53 or ridges are formed on the endless track device 15, extend longitudinally, and are opposite to the reinforcing ridges 50. Also, a mesh sheet 54 of fiber is disposed between the engaging teeth 52 and each of the guide projections 53.

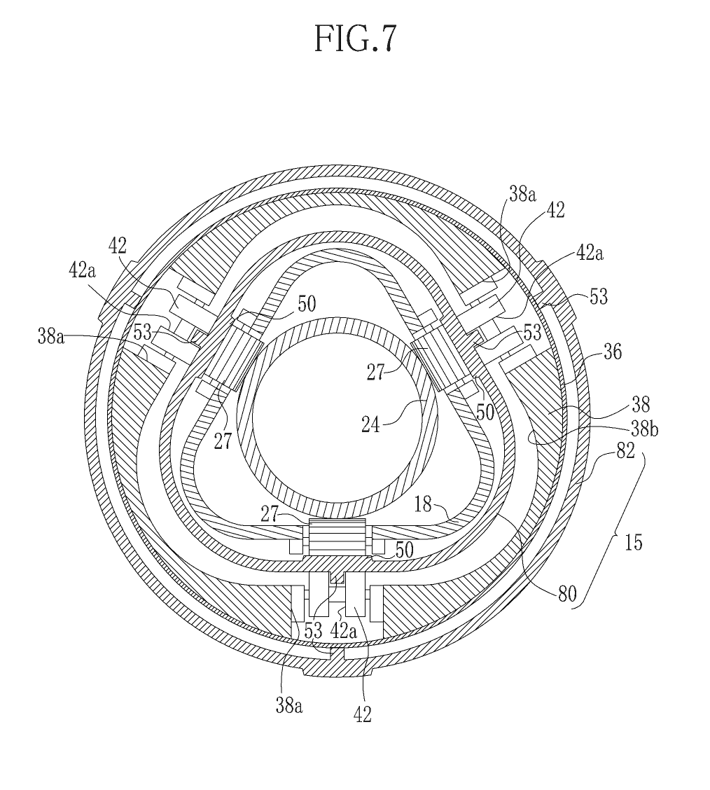

The endless track device 15 is used in the toroidal shape in FIG. 5. The three reinforcing ridges 50 are nipped between the roller wheels 27 and the idler rollers 42. See FIG. 7. The roller wheels 27 are meshed with the engaging teeth 52. Rotation of the roller wheels 27 is transmitted directly to the endless track device 15 by the engaging teeth 52. The endless track device 15 can turn around efficiently in the axial direction. The reinforcing ridges 50 and also the mesh sheet 54 are in the multi-layer form. The engaging teeth 52 in the endless track device 15 can have sufficient mechanical strength even upon receiving driving force directly from the roller wheels 27, because the engaging teeth 52 do not deform or the endless track device 15 does not break. Also, the membrane wall 51 disposed beside the reinforcing ridges 50 is effective in reducing resistance of the endless track device 15 during passage between the drive and barrel units 16 and 17.

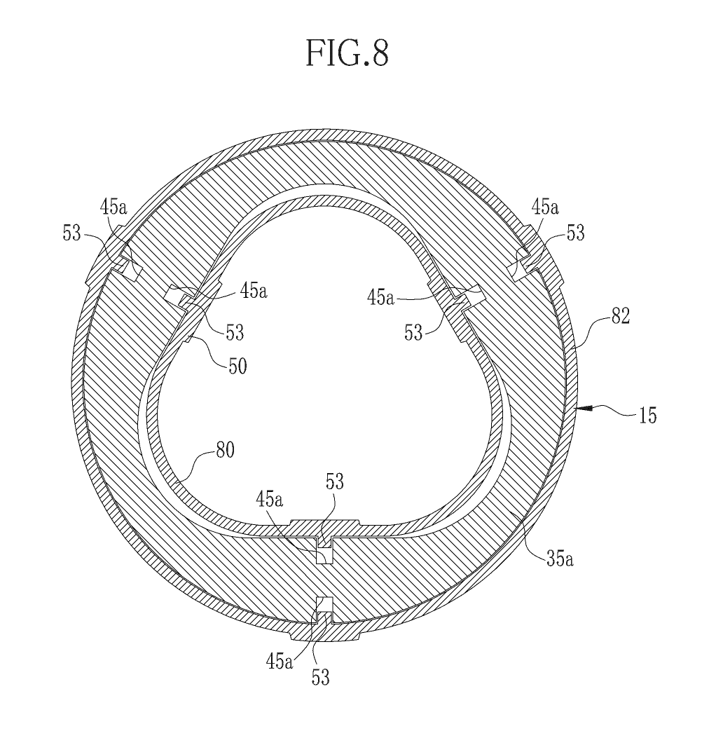

Roller grooves 42a are formed in respectively the idler rollers 42 at the center. In FIGS. 7 and 8, the guide projections 53 are engaged with the roller grooves 42a, and also with the guide grooves 45a and 45b of the support rings 35a and 35b. The guide projections 53 are effective in stabilizing the path of the movement, as the endless track device 15 can be prevented from shifting in a zigzag manner while moved in the axial direction. The cover sheet 36 and the barrel sleeve 38 do not have grooves for engagement with the guide projections 53. A contact width of each of the guide projections 53 to those is small. A clearance space is defined with the endless track device 15 adjacently to a portion of contact of the guide projections 53. Thus, friction in movement of the endless track device 15 is reduced without stiction phenomenon to the cover sheet 36 or the barrel sleeve 38.

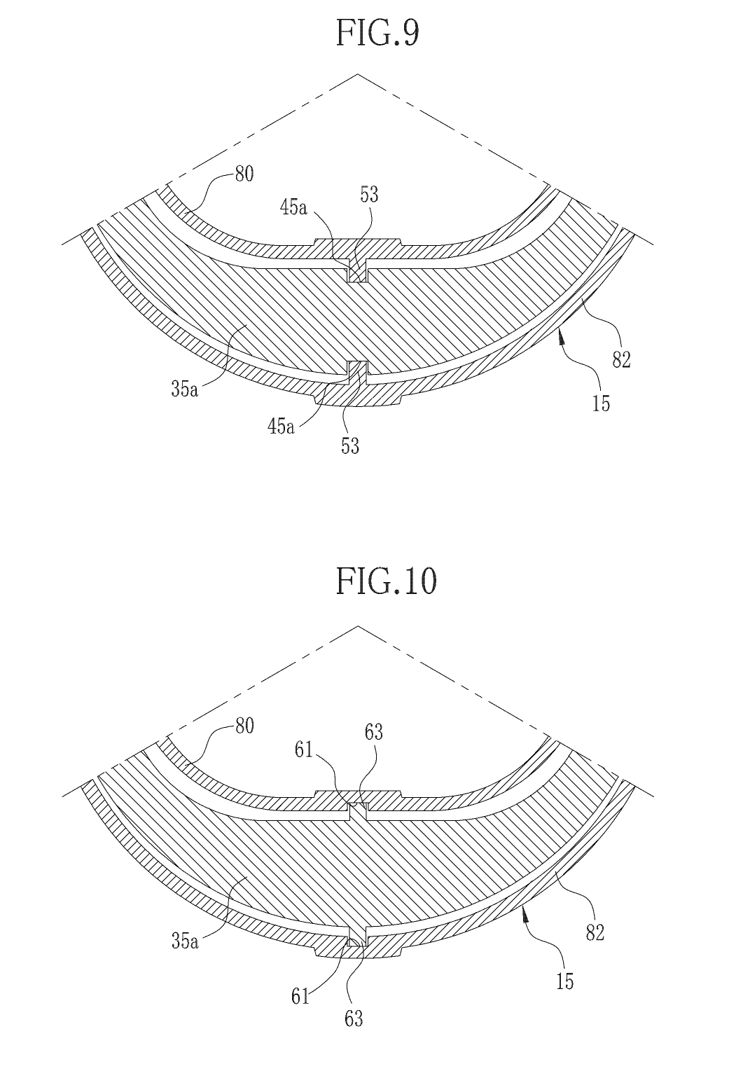

It is preferable as illustrated in FIG. 9 to determine a height of the guide projections 53 of the endless track device 15 larger than a depth of the guide grooves 45a and 45b formed in the support rings 35a and 35b. Also, guide grooves of a small depth can be formed in an inner surface 38b of the barrel sleeve 38 for contact with the guide projections 53 of the endless track device 15 in a similar manner to the guide grooves 45a and 45b. Guide grooves of a small depth can be formed in an outer surface of the cover sheet 36 similarly.

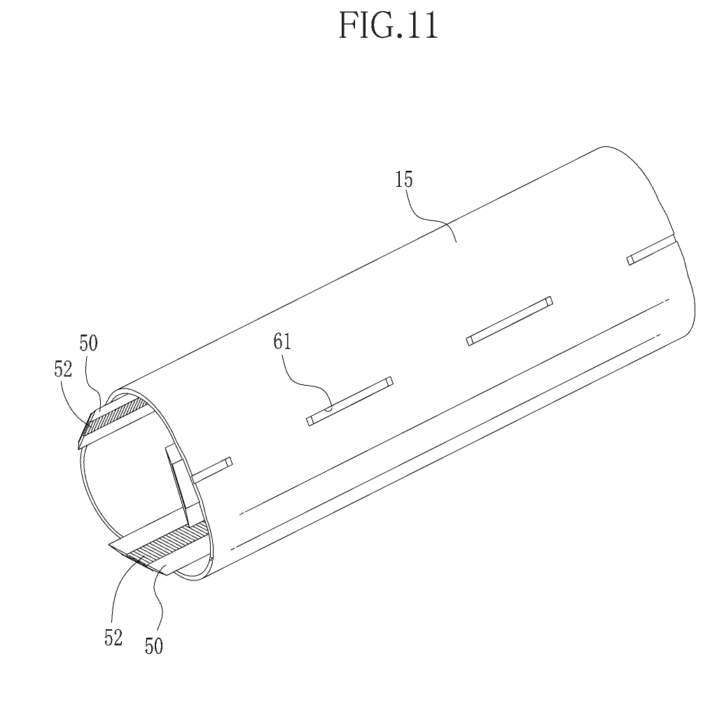

In FIG. 10, a variant structure is illustrated. Three guide grooves 61 are formed in the endless track device 15. Three guide projections 63 are formed on each of the support rings 35a and 35b. In FIG. 11, furthermore, three series of plural guide groove openings 61 are formed in the endless track device 15. Preferably, inclined surfaces can be formed at end portions of each of the guide groove openings 61 in a short form.

Furthermore, a surface of the membrane wall 51 of the endless track device 15 with the guide projections 53 can be processed by processing for reducing friction or processing for preventing a stiction phenomenon. Examples of the processing include forming a great number of ridges considerably smaller than the guide projections 53, processing of matte finish (rough surface finish), coating for smoothness with fluorocarbon resin, or the like.

Note that a series of a large number of small projections in the axial direction can be formed in place of each of the guide projections 53 described above.

In the above embodiment, each one of the guide projections 53 is formed on one of the three side areas of the endless track device 15. However, two or more of the guide projections 53 can be formed on each one of the side areas and arranged to extend in parallel. Also, plural series of a large number of small projections can be formed on each side areas of the endless track device 15. For such structures, guide grooves in a number according to the number of the guide projections are formed in place of the guide grooves 45a and 45b described above.

The operation of the above embodiment is described now. In FIG. 1, the propulsion assembly 2 is mounted on the endoscope in a state of protruding a distal end of the tip device 3 partially. A special screw driving device is used for mounting the propulsion assembly 2. The clamping sleeve 20 of a clamping mechanism is rotated by the screw driving device in the clockwise direction. The clamping sleeve 20 is helically engaged with a female thread formed inside the support sleeve 18 on the distal side. Rotation of the clamping sleeve 20 in the clockwise direction shifts the clamping sleeve 20 in the inward direction or proximal direction. The tapered end surface 20a presses the sealing device 21 or C-ring. A tapered surface on a distal side of the sealing device 21 is pressed by the tapered end surface 20a to deform the sealing device 21 to decrease its diameter. The tip device 3 is squeezed by the sealing device 21 inside the support sleeve 18 upon the deformation. The propulsion assembly 2 is fastened to the tip device 3 reliably.

The wire sheath 12 extending from the proximal end of the propulsion assembly 2 is positioned along the outer surface of the steering device and the flexible device of the endoscope. Plural indicia are disposed on the wire sheath 12 equidistantly from one another, and indicate positions of attachment of the adhesive tape 4. The wire sheath 12 is attached to the steering device and the flexible device by use of the adhesive tape 4 according to the indicia. The key coupling device 13 at the proximal end of the wire sheath is plugged to the rotating coupling 14 for connection to the actuating apparatus 10, which is powered.

The actuating apparatus 10 checks whether the key coupling device 13 is plugged to the rotating coupling 14 or not upon powering. If it is judged that the plugging is improper or if the plugging is not detected, alarm information is emitted, for example, alarm sound or a visible alarm signal with light. If it is judged that the plugging is proper, a sensor in the rotating coupling 14 reads type information of the propulsion assembly 2 from a signal region disposed on a bridge portion of the key coupling device 13. According to the type information, the actuating apparatus 10 automatically determines a rotational speed of the wire devices 30a and 30b and a value of a torque limiter, and prevents the wire devices 30a and 30b from operating at too high a speed or torque.

When the power source is turned on, the actuating apparatus 10 receives type information of the endoscope in connection with the processing apparatus 8 in a form of an output signal. The actuating apparatus 10 includes an inner storage medium. The actuating apparatus 10 recognizes the type information of the endoscope for use and type information of the propulsion assembly 2 by referring to table data stored in the storage medium. The table data is data of types of the endoscope and usable types of the propulsion assembly 2 in association with the endoscope types.

For example, a shiftable range of the sealing device 21 is determined according to the type information of the propulsion assembly 2. An outer diameter of the tip device 3 is determined according to the type information of the endoscope. It is possible promptly to check whether the propulsion assembly 2 can be properly used in connection with the tip device 3 of the endoscope. If it is judged that a combination of the propulsion assembly 2 with the tip device 3 is improper, an alarm signal is generated, for example, alarm sound or visible alarm sign of light with an alarm lamp. Also, operation of the propulsion assembly 2 may be inhibited. Those functions can prevent occurrence of accidents.

When a foot switch 11 in connection with the actuating apparatus 10 is depressed, the motors in the actuating apparatus 10 rotate to apply torque to the wire devices 30a and 30b. The coupling gears 32a and 32b are caused to rotate, so that the spur gear teeth 24b meshed with the first coupling gear 32a are rotated with the drive sleeve 24. The second coupling gear 32b rotates in a direction opposite to that of the first coupling gear 32a. Rotation of the second coupling gear 32b is directly transmitted to the first coupling gear 32a. Thus, the motors in the actuating apparatus 10 can be utilized to rotate the drive sleeve 24.

When the worm gear teeth 24a of the drive sleeve 24 rotate, the roller wheels 27, or worm wheels with helical gear teeth, rotate in the same direction about respectively the gear shaft 27a. The endless track device 15 is tensioned between the teeth of the roller wheels 27 and the idler rollers 42 of the roller mechanisms 40. The idler rollers 42 are caused to rotate by the roller wheels 27 to move the endless track device 15 endlessly in the axial direction of the drive sleeve 24.

In FIG. 5, the roller wheels 27 rotate in the clockwise direction. The idler rollers 42 rotate in the counterclockwise direction. A return run 80 of the endless track device 15 inside the barrel unit 17 moves from the proximal side to the distal side. A working run 82 of the endless track device 15 outside the barrel unit 17 moves from the distal side to the proximal side. Thus, the endless track device 15 endlessly turns around in the direction Y.

The working run 82 of the endless track device 15 contacts a wall of the large intestine in entry of the endoscope with the propulsion assembly 2 in the gastrointestinal tract. While the endless track device 15 endlessly moves, propulsion force for advancing the tip device 3 is obtained, in other words, force for pressing the wall of the large intestine in the proximal direction is obtained.

During the distal movement of the endoscope, foreign material stuck on the working run 82 of the endless track device 15 may move toward the position of the return run 80 after passing the proximal end of the barrel unit 17. However, the flange edge of the proximal cover flange 19b is positioned very close to the endless track device 15 and prevents the foreign material from internal jamming. Also, the proximal cover flange 19b prevents tissue of a body part from internal jamming together with the endless track device 15. Note that during the proximal movement of the endoscope, the flange edge of the distal cover flange 19a operates in the same manner for protection.

As the endless track device 15 endlessly moves between the roller wheels 27 and the idler rollers 42, the reinforcing ridges 50 in a layered structure are formed on the endless track device 15 for mechanical strength and durability to protect the engaging teeth 52 from abrasion and damage. It is likely that torsional pressure is applied to the endless track device 15 by incidental contact of foreign material or obstacle. However, the guide projections 53 positioned opposite to the engaging teeth 52 of the reinforcing ridges 50 are engaged with the roller grooves 42a of the idler rollers 42. The engaging teeth 52 will not be disengaged from the roller wheels 27. The guide projections 53 move in engagement with the guide grooves 45a and 45b formed in the support rings 35a and 35b. Thus, the endless track device 15 can return to the roller wheels 27 and the idler rollers 42 without large offset due to an obstacle, because the endless track device 15 can be oriented appropriately.

The endless track device 15 moves endlessly while the working run 82 contacts the cover sheet 36 and the return run 80 contacts the barrel sleeve 38. However, no grooves are formed in the cover sheet 36 or the barrel sleeve 38 for engagement with the guide projections 53. A contact width of each of the guide projections 53 to those is small. A clearance space is defined between the endless track device 15 and the cover sheet 36 or the barrel sleeve 38 adjacently to a portion of contact of the guide projections 53. Thus, friction in movement of the endless track device 15 is reduced without stiction phenomenon to the cover sheet 36 or the barrel sleeve 38. Breakage of the endless track device 15 can be prevented. Also, load to the driving mechanism (drive unit) for turning around the endless track device 15 can be reduced. The same effect can be obtained in the variant structures illustrated in FIGS. 7-11.

If the operator wishes to remove the propulsion assembly 2 from the tip device 3, the clamping sleeve 20 is rotated in the counterclockwise direction by use of the screw driving device. The clamping sleeve 20 shifts in an outward direction by rotating, and releases the sealing device 21 from being pressed. The sealing device 21 is enlarged by its resiliency to separate its inner surface from an outer surface of the tip device 3. The propulsion assembly 2 can be removed from the endoscope easily.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A propulsion assembly for an endoscope, comprising:
a support sleeve for mounting on a tip portion of an elongated tube of said endoscope;
a barrel unit disposed around said support sleeve;
an endless track device, disposed to extend along inner and outer surfaces of said barrel unit, for endlessly moving in an axial direction of said elongated tube in contact with a wall of a body cavity, for propulsion of said tip portion;
a plurality of roller wheels, disposed on said support sleeve, for driving said endless track device by engagement therewith;
a plurality of idler rollers, disposed on said inner surface of said barrel unit in a rotatable manner, for applying tension to said endless track device in cooperation with said roller wheels;
at least one guide projection formed on a first one of said endless track device and said barrel unit;
at least one guide groove, formed in a second one of said endless track device and said barrel unit, for receiving said guide projection, to guide said endless track device on said barrel unit in said axial direction.

2. A propulsion assembly as defined in claim 1, further comprising:
a drive sleeve, supported around said support sleeve, for rotating upon application of torque;
worm gear teeth, formed around said drive sleeve, for rotating said roller wheels.

3. A propulsion assembly as defined in claim 2, wherein said roller wheels have helical gear teeth.

4. A propulsion assembly as defined in claim 2, wherein said guide projection is formed on said endless track device, and said guide groove is formed in said barrel unit.

5. A propulsion assembly as defined in claim 4, wherein said barrel unit includes proximal and distal end surfaces, positioned opposite to one another with reference to said axial direction, for inverting said endless track device;
said guide groove is formed in at least one of said proximal and distal end surfaces.

6. A propulsion assembly as defined in claim 5, wherein said barrel unit includes:
a barrel sleeve; and
a support ring, attached to one end of said barrel sleeve in said axial direction, having said proximal or distal end surface, said guide groove being formed therein.

7. A propulsion assembly as defined in claim 4, wherein a depth of said guide groove is smaller than a height of said guide projection.

8. A propulsion assembly as defined in claim 4, further comprising a roller groove, formed in each of said idler rollers, for receiving said guide projection.

9. A propulsion assembly as defined in claim 2, wherein said at least one guide groove has plural guide groove openings arranged in series.

10. A propulsion assembly as defined in claim 2, wherein said guide projection is formed on said barrel unit, and said guide groove is formed in said endless track device.

11. A propulsion assembly as defined in claim 2, wherein said endless track device is toroidal.
